(19) United States
(12) Patent Application Publication
(10) Pub. No.: US 2006/0270885 A1
(43) Pub. Date: Nov. 30, 2006

(54) Normal heptane isomerization

(76) Inventors: Christopher C. Boyer, Houston, TX (US); Mitchell E. Loescher, Houston, TX (US); Jinsuo Xu, Hillsborough, NJ (US); Frits M. Dautzenberg, San Diego, CA (US)

Correspondence Address: KENNETH H. JOHNSON, P.O. BOX 630708, HOUSTON, TX, 77263, US

(21) Appl. No.: 11/149,504

(22) Filed: May 31, 2005

(51) Int. Cl. C07C 5/13

(52) U.S. Cl. 585/741

(57) ABSTRACT

A process for the isomerization of heptane preferably contained within a naphtha stream is disclosed wherein the naphtha is stripped of the butanes and the pentanes and hexanes are removed for isomerization. The heptanes and heavier are fed to a distillation column reactor containing an isomerization catalyst where the normal heptane is isomerized to mono and di branched heptane and removed as overheads. The cyclic heptanes and heavier are removed as bottoms and for feed to a catalytic reforming process.

### BACKGROUND OF THE INVENTION

1. Field of the Invention

The present invention relates to a process for concurrently fractionating a normal heptane containing feed and isomerizing the normal heptane to branched heptane. More particularly the invention relates to a process in which the normal heptane is contained in a naphtha stream.

2. Related Information

Petroleum distillate streams contain a variety of organic chemical components. Generally the streams are defined by their boiling ranges which determine the compositions. The processing of the streams also affects the composition. For instance, products from either catalytic cracking or thermal cracking processes contain high concentrations of olefinic materials as well as saturated (alkanes) materials and polyunsaturated compounds (e.g., diolefins). Additionally, these components may be any of the various isomers of the compounds.

Reformed naphtha or reformate generally requires no further treatment except perhaps distillation or solvent extraction for valuable aromatic product removal. However, reforming of the C₇ fraction of the naphtha results in the formation of aromatics, especially benzene, the content, of which in gasoline is being restricted. Isomerization of the C₇ portion is thus attractive to take the light fraction of the reformer feed to make high octane fuel with less aromatics. However, the isomerization of the C₇'s has resulted in the fouling of the isomerization catalyst due to coking caused by cracking of the longer chain compounds. Thus, isomerization has been limited in the past to the lighter C6 fraction.

The advantages of using the isomerization process in a refinery include:

(1) removing the C₇ cut reduces the amount of benzene produced in the reformer and eliminates the need for a benzene removal unit downstream of the reformer;

(2) removing the C₇ cut allows the reformer to operate at conditions that have improved yields;

(3) gives more flexibility on the cut that is sent to the C₅/C₆ isomerization process;

(4) increases the hydrogen/feed production because the C₇ paraffins contribute very little hydrogen;

(5) improves the octane of the C₇ cut without producing aromatics which reduces the aromatic content in the gasoline blend; and

(6) either the C₅/C₆ splitter or the C₇ splitter can be shut down and by passed without disrupting other refinery operations since the reformer can operate with or without theses streams and the C₇ splitter can handle the C₅/C₆ cut.

### SUMMARY OF THE INVENTION

Briefly a preferred embodiment of the present invention is a C₇ isomerization process using a catalytic distillation reactor as a C7 isomerization splitter. Briefly the invention is a process for the isomerization of normal heptane contained within hydrocarbon stream comprising the steps of:

(a) concurrently:
(i) contacting normal heptane contained in a hydrocarbon feed containing normal heptane and heavier material with an isomerization catalyst under conditions of temperature and pressure to isomerize normal heptane to branched heptane and
(ii) separating the branched heptane from the normal heptane and heavier material by fractional distillation; and

(b) withdrawing the branched heptane and the heavier material separately.

In a preferred embodiment the reaction mixture is fractionated to maintain a selected fraction comprising normal heptanes and not the heavies portion of a naphtha stream in the distillation reaction zone (the zone containing the isomerization catalyst) to selectively isomerize at least a portion of the normal heptane to branched heptanes to form a reaction mixture.

In another preferred embodiment light hydrotreated straight naphtha, naphtha from a hydrocracker or hydrotreated coker naphtha are sent first to a dehexanizer (cut point 160-1 70° F.) where the C₅/C₆ is taken as overheads and the bottom product is sent to a C₇ isomerization splitter. A reaction zone is located above the feed point and at a point where the n-C₇ bulges. The C₇ isomerized product leaves the column as overheads and heavy reformate feed leaves as bottoms. The reaction zone may contain catalyst held in place by structured packing, or it may include a slurry bed at the bottom of a second column in an articulated column system. The reaction zone may also be in a reactor outside the column fed by a sidedraw on the column with the products returning to the main column.

In addition to the preferred process for isomerizing normal heptane, other alkanes are isomerized by the present process, in particular the alkanes found within the naphtha cut fed to the catalytic distillation reactor, preferably the C₄-C₈ alkanes, including branched alkanes capable of further branching under the conditions of the isomerization, e.g., methyl hexane which can be isomerized to dimethyl pentane. Thus, in the broader sense the present invention is a process for the isomerization of alkanes contained within hydrocarbon stream comprising the steps of concurrently contacting an alkane having a first skeletal configuration and contained in a hydrocarbon feed with an isomerization catalyst under conditions of temperature and pressure to isomerize the alkane of said first skeletal configuration to a second more highly branched skeletal configuration; separating the second more highly branched alkane from the alkane having said first configuration by fractional distillation; and recovering the second more highly branched alkane.

The typical feed to a reformer has between 8 and 17 wt. % paraffin C₇'s of which 45 to 60 wt. % are n-heptane and 30-42 wt. % are methyl hexane. A C₇ cut representing about 10 to 20% of the current reformer feed can be sent through the isomerization process.

For the purposes of the present invention, the term “catalytic distillation” includes reactive distillation and any other process of concurrent reaction and fractional distillation in a column, i.e., a distillation column reactor, regardless of the designation applied thereto.

### BRIEF DESCRIPTION OF THE DRAWING

FIG. 1 is a simplified flow diagram in schematic form of the invention.

FIG. 2 is comparison of naphtha cracking between two isomerization catalysts.

### DETAILED DESCRIPTION OF THE PREFERRED EMBODIMENT

The particular advantages of the present process using a distillation column reactor for the C₇ isomerization are:

(1) reduction in equipment count because the C₇ splitter, reactor and C₇ paraffin separator are all contained in one unit;

(2) the C7 boiling point properties match the temperature and pressure conditions required for the isomerization reaction;

(3) distillation allows for recycle both mono branched and normal C₇'s back to the reaction zone which increases the yield of higher di-branched product compared to units which only recycle the normal paraffins;

(4) in catalytic distillation the cyclic C₇'s are still part of the bottom product which is sent to the reformer as compared to a traditional process where the cyclics have to be cut out with the normal C₇'s to be sent to the isomerization unit which results in an overall octane disadvantage, or in the alternative a large fraction of the normal heptane would have to be fed to the reformer;

(5) the catalytic distillation process provides a longer catalyst life than a fixed bed process because it has improved wetting characteristics and continually washes heavy species off the catalyst and removes them from the bottom of the column thus preventing coke formation; and

(6) the catalytic distillation process gives better yield, i.e., produces less over cracked products because the lighter species are removed from the reactor by distillation giving them a shorter residence time than the normal heptane, consequently thes primary products are less likely to undergo cracking.

The present process is preferably carried out in a catalytic distillation reactor. Preferably the reactor is operated in a manner to hold the normal heptane in the catalyst, which facilitates the isomerization and reduces the amount of heavier component s of the feed in contact with the catalyst and consequently reduces the potential for cracking of the heavies. In the case of a naphtha feed, introduced into a catalytic distillation reactor with the catalyst prepared as a distillation structure and arranged in the column, the feed point is conveniently immediately below the catalyst position. The column is preferably operated to maintain the C₇ portion of the feed in the catalyst.

Essentially the distillation column reactor is operated as a splitter with the C₇ and lighter material going overhead and the C₈ and heavier going out as bottoms. In the current process the temperature is controlled by operating the reactor at a given pressure to allow partial vaporization of the reaction mixture. The exothermic heat of reaction is thus dissipated by the latent heat of vaporization of the mixture. The vaporized portion is taken as overheads and the condensible material condensed and returned to the column as reflux.

The downward flowing liquid causes additional condensation within the reactor as is normal in any distillation. The contact of the condensing liquid within the column provides excellent mass transfer within the reaction liquid and concurrent transfer of the reaction mixture to the catalytic sites. A further benefit that this reaction may gain from catalytic distillation is the washing effect that the internal reflux provides to the catalyst thereby reducing polymer build up and coking. Internal reflux may vary over the range of 0.2 to 20 L/D (wt. liquid just below the catalyst bed/wt. distillate) which gives excellent results.

A particularly unexpected benefit of the present process centers on the combined reaction distillation going on in the column. in addition to the naphtha comprises a mixture of organic aromatic compounds boiling over a range. The product from the isomerization can be tailored by adjusting the temperature in the column to fractionate the naphtha feed concurrently with the isomerization of the normal C7 and the distillation of the isomerization product. Any cut can be made that is within the capacity of the equipment. For example, the light end of the naphtha along with the branched heptanes can be taken overhead, heavies such as octane taken as bottoms and a high concentration of normal heptane maintained in the portion of the column containing the catalytic distillation structure. The location of the catalyst bed can also be tailored for optimum results.

As with any distillation there is both a vapor phase and a liquid phase, e.g., the internal reflux. The present process operates at overhead pressure of said distillation column reactor in the range between 0 and 350 psig, preferably 250 or less suitable 35 to 120 psig and temperatures in said distillation reaction bottoms zone in the range of 100 to 500° F, preferably 150 to 400° F., more preferably 212 to 374° F. The isomerization process may be carried out either in the presence or absence of hydrogen. The mole ratio of hydrogen to hydrocarbon is preferably in the range of 0.01:1 to 10:1.

In order to maintain normal heptane in a C7 naphtha cut within the catalyst bed, for example, the pressure can be at 75 psig to maintain an overhead temperature of about 275° F., mid reflux of about 300° F. and a bottoms temperature of about 400° F. The temperature in the catalyst bed would be around 270° F.

The feed weight hourly space velocity (WHSV), which is herein understood to mean the unit weight of feed per hour entering the reaction distillation column per unit weight of catalyst in the catalytic distillation structures, may vary over a very wide range within the other condition perimeters, e.g., 0.1 to 35.

The composition of untreated naphtha as it comes from the crude still, or straight run naphtha, is primarily influenced by the crude source. Naphthas from paraffinic crude sources have more saturated straight chain or cyclic compounds. As a general rule most of the “sweet” (low sulfur) crudes and naphthas are paraffinic. The naphthenic crudes contain more unsaturates and cyclic and polycylic compounds. The higher sulfur content crudes tend to be naphthenic. Treatment of the different straight run naphthas in the present process may be slightly different depending upon their composition due to crude source.

Catalysts which are useful for the isomerization of C₇'s include non-zeolitic catalyst as disclosed in U.S. Pat. Nos. 5,648,589, 6,706,659 and 6,767,859; and zeolites as disclosed in U.S. Pat. Nos. 6,124,516 and 6,140,547. Sulfonated zirconia oxide catalysts developed by Sudchemie have also been shown to be useful.

A preferred catalyst group for the present isomerization comprises non-zeolite catalytic compounds represented by the generalized formula:
R₁/R₄/R₂—R₃
wherein:
R₁ is a metal or metal alloy or bimetallic system;
R₂ is any metal dopant;
R₃ is a metallic oxide or mixtures of any metallic oxide;
R₄ is selected from WOₓ, MoOₓ, SO₄²— or PO₄³⁻; and
x is a whole or fractional number between and including 2 and 3.
   Preferably:

R₁ is selected from: a Group VIII noble metal or a combination of Group VIII noble metals; such as platinum, palladium, iridium, rhodium, nickel, cobalt or a combination thereof or a Pt—Sn, Pt—Pd, or Pt—Ga alloy, Pt—Ni alloy or bimetallic system:

R₂ is selected from the group Al³⁺, Ga^{3+, Ce}₄₊, Sb⁵⁺, Sc³⁺, Mg²⁺, Co²⁺, Fe³⁺, Cr³⁺, Y³⁺Si⁴⁺, and In³⁺;

R₃ is selected from the group zirconium oxide, titanium oxide, tin oxide, ferric oxide, cerium oxide or mixtures thereof;

R₄ is selected from SO₄²⁻, WOₓ, MoOₓ, PO₄³⁻, W₂₀O₅₈, W₁₀O₂₉ and anions and mixtures thereof; and

the ratio of metal dopant to metal in the oxide may be less than or equal to about 0.20, such as, less than or equal to about 0.05.

The catalyst may be placed in various configurations for conducting the isomerization and separations of the invention, such as, a separate reactor outside of a distillation column with a sidedraw for feed and the product being returned to the column. Preferably the catalyst is used in a distillation column reactor where it may be placed as a distillation structure as describe below or in a slurry bed at the bottom of the second column in an articulated column system.

When used in a distillation column reactor the catalyst may be prepared in the form of a catalytic distillation structure which functions as catalyst and as mass transfer medium. The catalyst is suitably supported and spaced within the column to act as a catalytic distillation structure. A variety of catalyst structures for this use are disclosed in U.S. Pat. Nos. 4,443,559; 4,536,373; 5,057,468; 5,130,102; 5,133,942; 5,189,001; 5,262,012; 5,266,546; 5,348,710; 5,431,890; and 5,730,843 which are incorporated herein by reference.

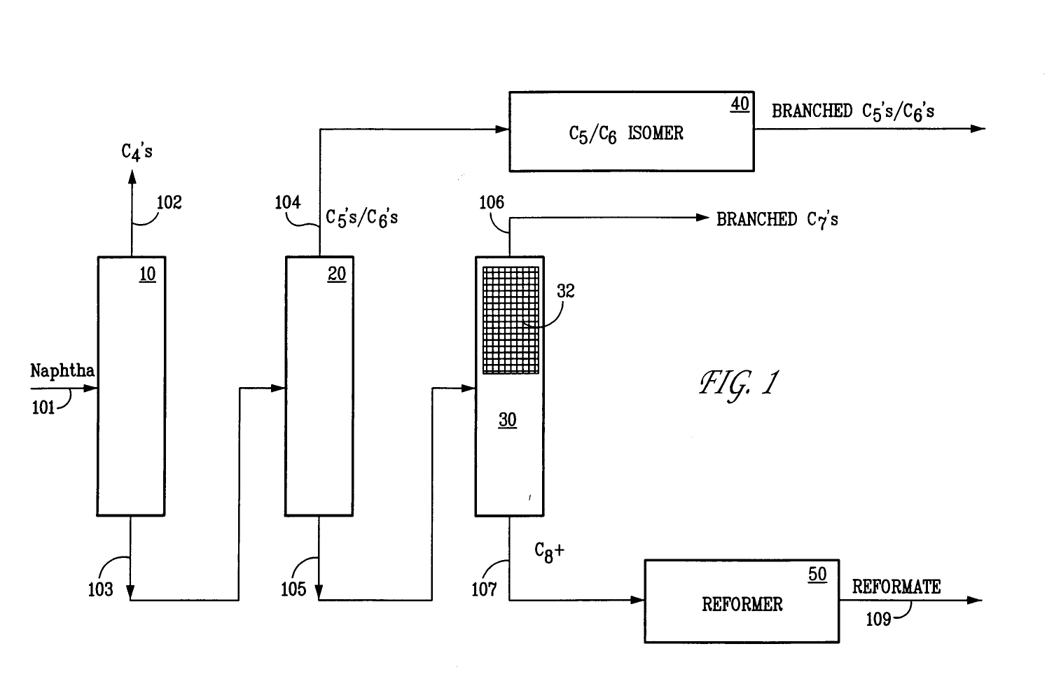

Referring now to the FIG. 1 a simplified flow diagram of the preferred process is shown. The naphtha, either straight run or hydrotreated cracked naphtha(i.e, FCCU, coker or visbreaker ), is first fed to a debutanizer 10 via flow line 101 where the C₄ and lighter material is taken as overheads via flow line 102 and the C₅ and heavier material is taken as bottoms via flow line 103. The C₅ and heavier material is fed to a naphtha 20 where the C₅ and C₆ material are taken as overheads via flow line 104 and the C₇ and heavier material are taken as bottoms via flow line 107. The C₅/C₆ are fed to a C₅/C₆ isomerization 40 unit where the C₅'s and C₆'s are isomerized to branched paraffins. The C₇ and heavier material are fed to a distillation column reactor 30 containing a bed 32 of isomerization catalyst. The C₇ material is boiled upward into the bed where the normal heptane is isomerized to branched heptanes. Some of the mono branched are isomerized further to multi branched heptanes. The isomerized C₇'s are removed as overheads via flow line 106. The bottoms, containing the C₈'s and heavier and cyclic C₇'s are removed as bottoms via flow line 107 and fed to a standard catalytic reforming unit 50 to upgrade the octane. All of the C₅ and heavier material may be utilized as desired in the gasoline blend.

### EXAMPLE 1

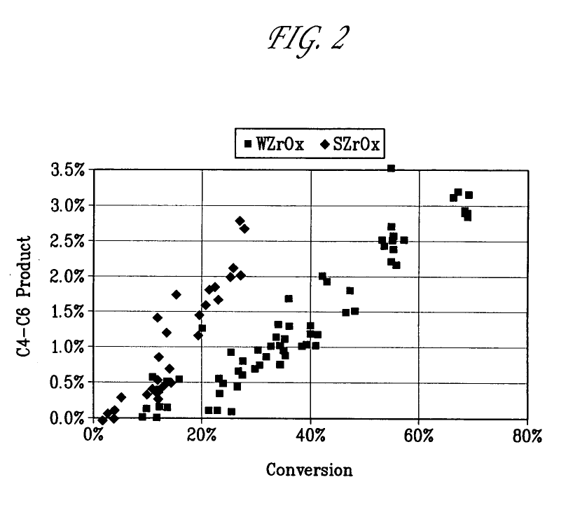

A tungstated zirconia according to U.S. Pat. No. 6,767,859 demonstrated lower cracking than the sulfated zirconia of Example 2 by a factor of four as shown in FIG. 2. In fixed evaluation, the sulfated zirconia catalyst was restricted to conversion of less than 30% to prevent catalyst fouling is a matter of hours, whereas the tungstated zirconia would have catalyst stability at conversion of about 45%. In a naphtha stream treated to isomerize the normal heptane, cracking is undesirable since this represents a net loss of naphtha. However, both catalyst will preform in an improved manner in the catalytic distillation mode.

The fixed bed reactor was a ½″ tubing (0.4″ ID) loaded with 20 g of catalyst. The catalyst exudates (˜1.6 mm diameter) were not crushed but were broken so that their length did not exceed 3 mm. The catalyst was riot diluted with an inert material. The reactor consisted of a 10 ml preheat section followed by a 20 ml reaction zone. Thermocouples measured the temperature at the inlet and outlets of the reaction zone. Separate heaters controlled the preheat section and reactor section temperatures. Pressure was controlled by a manual back pressure regulator.

The start up procedure for fixed bed evaluations:

1. Heat Catalyst in Reactor:

a. Load 20 g catalyst into reactor.

b. Start air flowing to 100 sccm. Ambient pressure.

c. Heat reactor to 250° F. (120° C.) at rate<10° F./min. and hold for 1 hour.

d. Heat reactor to 840° F. (450° C.) at rate<10° F./min and then hold in air for 1.5 hours.

e. Switch gas to N₂ at 30 sccm and allow catalyst to cool to 220° F.

2. Reduce Catalyst:

a. Continue N₂ flow and heat to 600° F. Set Pressure at ˜20 psig.

b. When temps reach 600° F., switch to H₂ gas flow at 175 sccm for 3 hours.

c. Cool reactor to 350° F. with H₂.

3. Start Hydrocarbons:

a. Increase pressure to 200 psig and keep at 350° F.

b. Keep H₂ flow rate at 175 sccm.

c. Start hydrocarbon flow at 1 ml/min.

Liquid samples were taken of the n-heptane being fed to the reactor and of the liquid phase effluent from the reactor after the product was cooled to room temperature. Samples were taken every 6 hours at the start of the run and later every 3 hours. The residence time in the reactor and tubing to the sample point was about ½ hour at a WHSV of 2.1. Changes in conditions were made at least two hours before a sample was taken. A GC analyzed the samples and identified species in the C₄ to C₈ range. Cracked species in the C₁ to C₃ range were not observed. Some of the lighter components may also have been concentrated in the vapor phase. It was assumed that all C₄ and heavier material was only in the liquid phase.

### EXAMPLE 2

A 1″ diameter distillation column reactor was loaded with 10 feet of Sud Chemie isomerization catalyst. Normal heptane was fed to the column. Conditions and results are given in TABLE I below. The conversion used in the run was about 40%, well above that indicated in the fixed bed evaluations of Example 1.

TABLE I

Run time, hrs

| Conditions | 143 | 239 |
|---|---|---|
| Pressure, psig | 100 | 100 |
| average bed temp., ° F. | 343 | 348 |
| H2, SCFH | 10 | 5 |
| Feed, lb/hr | 1.4 | 1.5 |
| Reflux, lb/hr | 5.3 | 1.8 |
| Bottoms, lb/hr | 0.3 | 0.3 |
| Overheads, lb/hr | 1.0 | 1.0 |
| Vent, lbs/hr | 0.1 | 0.1 |
| nC7 WHSV, lb/lb cat/hr | 2.5 | 2.5 |
| nC₇ wt fraction in overheads | 56.7 | 60.2 |
| nC₇ wt fraction in bottoms | 92.9 | 93.3 |
| nC₇ wt fraction in feed | 99.4 | 99.5 |
| C₇+ HC in vent, mol fraction | 31.1 | 49.2 |
| nC₇ upflow conversion | 43.0 | 39.5 |
| H2/nC7 molar ratio | 2.4 | 1.2 |
| Yield, based on nC₇, % | 96.5 | 95.6 |
| Cracking, based on C₇, % | 3.5 | 4.4 |
| Mono branched selectivity, % | 70.2 | 70.4 |
| Di branched selectivity, % | 28.6 | 28.3 |
| Tri branched selectivity, % | 1.2 | 1.2 |

The invention claimed is:

1. A process for the isomerization of normal heptane contained within a hydrocarbon stream comprising the steps of:
(a) concurrently:
(i) contacting normal heptane contained in a hydrocarbon feed containing normal heptane and heavier material with an isomerization catalyst under conditions of temperature and pressure to isomerize normal heptane to branched heptane and
(ii) separating the branched heptane from the normal heptane and heavier material by fractional distillation; and
(b) withdrawing the branched heptane and the heavier material separately.

2. The process according to claim 1 wherein the conditions of temperature and pressure are such that the normal heptane is maintained in contact with the isomerization catalyst so that essentially all of the normal heptane is converted to branched heptane.

3. The process according to claim 1 wherein a portion of the branched heptane formed is isomerized to di branched heptane.

4. The process according to claim 1 wherein said hydrocarbon feed comprises a naphtha stream.

5. The process according to claim 1 wherein the naphtha stream contains butanes and the butanes are removed prior to feeding the naphtha to the isomerization.

6. The process according to claim 1 wherein the naphtha stream contains pentanes and hexanes and the pentanes and hexanes are removed prior to feeding the naphtha to the isomerization.

7. The process according to claim 1 wherein the isomerization catalyst comprises a compound of the generalized formula:
R₁/R₄/R₂—R₃
wherein:
R₁ is a metal or metal alloy or bimetallic system;
R₂ is any metal dopant;
R₃ is a metallic oxide or mixtures of any metallic oxide;
R₄ is selected from WOₓ, MoOₓ, SO₄²⁻ or PO₄³⁻; and
x is a whole or fractional number between and including 2 and 3.

8. The process according to claim 7 wherein
R₁ is a Group VIII noble metal or a combination of Group VIII noble metals;
R₂ is selected from the group consisting of Al³⁺, Ga³⁺, Ce⁴⁺, Sb⁵⁺, Sc³⁺, Md²⁺, Co²⁺, Fe³⁺, Cr³⁺, Y³⁺Si⁴⁺, and In³⁺;
R₃ is zirconium oxide, titanium oxide, tin oxide, ferric oxide, cerium oxide or mixtures thereof;
R₄ is selected from the group consisting of SO₄²⁻, WOₓ, MoOₓ, PO₄³⁻, W₂₀O₅₈, W₁₀O₂₉ and anions and mixtures thereof; and
the ratio of metal dopant to metal in the oxide may be less than or equal to about 0.20.

9. The process according to claim 8 wherein R₁ is platinum, palladium, iridium, rhodium, nickel, cobalt or a combination thereof.

10. The process according to claim 8 wherein R₁ is a Pt—Sn alloy, Pt—Pd alloy, Pt—Ga alloy, Pt—Ni alloy or bimetallic system thereof.

11. A process for the isomerization of normal heptane contained within a naphtha stream comprising the steps of:
(a) feeding a naphtha stream containing normal heptane and heavier material to a distillation column reactor containing a bed of isomerization catalyst;
(b) concurrently in said distillation column reactor,
(i) isomerizing a portion of the normal heptane to branched heptane and
(ii) separating the branched heptane from the normal heptane and heavier material by fractional distillation;
(c) withdrawing branched heptane as overhead product; and
(d) withdrawing the heavier material from the distillation column reactor as bottoms.

12. The process according to claim 1 1 wherein the distillation column reactor is operated such that the normal heptane is maintained in the bed of isomerization catalyst so that essentially all of the normal heptane is converted to branched heptane.

13. The process according to claim 11 wherein a portion of the branched heptane formed is isomerized to di branched heptane.

14. The process according to claim 11 wherein the naphtha also contains cyclic heptanes and the cyclic heptanes are removed in said bottoms.

15. The process according to claim 14 wherein said bottoms is fed to a catalytic reforming reactor.

16. The process according to claim 11 wherein the naphtha stream contains butanes and the butanes are removed in a debutanizer prior to feeding the naphtha to the distillation column reactor.

17. The process according to claim 11 wherein the naphtha stream contains pentanes and hexanes and the pentanes and hexanes are removed in a naphtha splitter prior to feeding the naphtha to the distillation column reactor.

18. The process according to claim 17 wherein the removed pentanes and hexanes are fed to a C5/C6 isomerization reactor where a portion of the normal pentanes and normal hexanes are converted to branched pentanes and branched hexanes respectively.

19. The process according to claim 11 wherein said distillation column reactor is operated under the conditions to (i) maintain a selected fraction comprising at least a portion of the normal heptane in the distillation reaction zone to selectively isomerize at least a portion thereof to branched heptanes and (ii) to exclude heavier material from the distillation reaction whereby cracking of heavier material is inhibited.

20. A process for the isomerization of heptanes contained in a naphtha stream comprising the steps of:
(a) feeding naphtha stream containing C₄'s, C₅'s, C₆'s, C₇'s and C₈'s and heavier compounds to a debutanizer wherein the C₄'s and lighter material are taken as a first overhead and the C₅'s and heavier are taken as a first bottoms;
(b) feeding the first bottoms to a naphtha splitter wherein the C₅'s and C₆'s are taken as a second overhead and the C₇'s and heavier material are taken as a second bottoms;
(c) feeding the second overheads to a C₅/C₆ isomerization reactor where the normal C5's and C6's are isomerized to branched C₅'s and C₆'s;
(d) feeding the second bottoms to a distillation column reactor containing a bed of isomerization catalyst;
(e) concurrently in the distillation column reactor,
(i) isomerizing a portion of the normal C₇'s to mono branched C₇'s and a portion of the mono branched C₇'s to di branched C₇'s, while maintaining the normal C₇'s within the catalyst bed and
(ii) separating the branched C₇'s from the C8's and any cyclic C₇'s;
(f) withdrawing the branched C₇'s from the distillation column reactor as a third overheads;
(g) withdrawing the C₈'s and cyclic C₇'s from the distillation column reactor as a third bottoms; and
(h) feeding the third bottoms to a catalytic reforming reactor to increase the octane.

21. A process for the isomerization of alkanes contained within hydrocarbon stream comprising the steps of:
(a) concurrently:
(i) contacting an alkane having a first skeletal configuration and contained in a hydrocarbon feed with an isomerization catalyst under conditions of temperature and pressure to isomerize the alkane of said first skeletal configuration to a second more highly branched skeletal configuration and
(ii) separating the second more highly branched alkane from the alkane having said first configuration by fractional distillation; and
(b) withdrawing the second more highly branched alkane.
